(19) United States
(12) Patent Application Publication
(10) Pub. No.: US 2017/0027629 A1
(43) Pub. Date: Feb. 2, 2017

(54) IMPLANTABLE BONE GRAFTING DEVICES, SYSTEMS, AND METHODS

(71) Applicants: Matthew Ackerman, Salt Lake City, UT (US); Vishal Bhola, Salt Lake City, UT (US); Michael Polei, Salt Lake City, UT (US); Nathaniel L.R. Rhodes, Salt Lake City, UT (US); Barbu Gociman, Salt Lake City, UT (US)

(72) Inventors: Matthew Ackerman, Salt Lake City, UT (US); Vishal Bhola, Salt Lake City, UT (US); Michael Polei, Salt Lake City, UT (US); Nathaniel L.R. Rhodes, Salt Lake City, UT (US); Barbu Gociman, Salt Lake City, UT (US)

(21) Appl. No.: 15/303,383

(22) PCT Filed: Apr. 9, 2015
(86) PCT No.: PCT/US15/25174

§ 371 Date: Oct. 11, 2016

Related U.S. Application Data
(60) Provisional application No. 61/995,446, filed on Apr. 9, 2014.

(51) Int. Cl. A61B 17/88; A61F 2/28

(52) U.S. Cl. CPC A61B 17/8808; A61F 2/2875; A61B 17/8805; A61F 2002/30593

(57) ABSTRACT

Implantable bone grafting devices, systems, and methods are disclosed and described. In one embodiment, a device may include a base, a lid, and at least one connector that connects the base and lid in a fixed relationship with a space therebetween. The space is configured as a reservoir to hold, and allow vascular access to, a bone grafting media. The bone grafting devices are configured for placement in a subject such that the bone grafting media reservoir substantially aligns in a common plane with a bone of the subject.

### FIELD OF TECHNOLOGY

The present technology relates to devices, systems, and associated methods for grafting bone media to a bone of a subject. Accordingly, invention embodiments involve the fields of biology, chemistry, pharmaceutical sciences, veterinary sciences, medicine, and other health sciences.

### BACKGROUND

Current surgical procedures related to bone repair and reshaping, such as both the reshaping and repair of maxilla-craniofacial defects, are often done using rigid, static, and artificial implant materials which require a high rate of revision surgeries. The two most commonly used rigid materials are Polyether ether ketone (PEEK), which is a colorless organic polymer thermoplastic in the polyaryletherketone (PEAK) family, and Titanium alloy. These methods are also very expensive and undesired by patients and physicians. Other methods of repair involve using bone cements often made from poly methyl-methacrylate (PMMA). Bone cements are inexpensive, but they are rigid and poorly approximate the damaged area. While bone cements offer a higher biocompatibility than other foreign materials, their high degree of rigidity often results in stress fracturing and the inability to reshape with patient physiological changes such as growth and development.

### BRIEF DESCRIPTION OF THE DRAWINGS

Invention features and advantages will be apparent from the detailed description which follows, and are further enhanced in conjunction with the accompanying drawings, which together illustrate, by way of example, various invention embodiments; and, wherein:

FIG. 1 is a cross-sectional view of an example invention embodiment;

FIG. 2 is a perspective view of a lid and base of an example invention embodiment;

FIG. 3 is a perspective view of the base of the example invention embodiment of FIG. 2

FIG. 4 is a side-by-side view of the inner surface of a lid and base of an example invention embodiment;

FIG. 5 is a side-by-side view of the outer surface of a lid and base of an example invention embodiment;

FIG. 6 is a perspective view of an example invention embodiment.

These figures are provided to illustrate various aspects certain invention embodiments and are not intended to be limiting in scope in terms of dimensions, materials, configurations, arrangements or proportions unless otherwise limited by the claims.

### DESCRIPTION OF EMBODIMENTS

Although the following detailed description contains many specifics for the purpose of illustration, a person of ordinary skill in the art will appreciate that many variations and alterations to the following details can be made and are considered to be included herein. Such alterations or variations may become apparent after a review of the present application. Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this specification and the appended claims, the singular forms “a,” “an” and “the” include plural referents unless the context clearly dictates otherwise. Thus, for example, reference to “a subject” includes a plurality of subjects.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term in this specification, like “comprising” or “including,” it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

Occurrences of the phrase “in one embodiment,” or “in one aspect,” herein do not necessarily all refer to the same embodiment or aspect.

As used herein the term “plane” refers to a two dimensional surface having a length and a width. In some embodiments, the plane can comprise a flat two dimensional surface. In other embodiments, the plane can comprise a curved surface. In yet other embodiments, the surface can have curved portions and flat portions or substantially curved portions and substantially flat portions.

As used herein a “therapeutic agent” or “therapeutic additive” can be used interchangeably refer to an agent that can have a beneficial or positive effect on a subject when administered to the subject in an appropriate or effective amount.

As used herein, an “effective amount” of an agent is an amount sufficient to accomplish a specified task or function desired of the agent. A “therapeutically effective amount” of a composition, drug, or agent refers to a non-toxic, but sufficient amount of the composition, drug, or agent, to achieve therapeutic results in treating or preventing a condition for which the composition, drug, or agent is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount or therapeutically effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986).

As used herein, the terms “release” and “release rate” are used interchangeably to refer to the discharge or liberation, or rate thereof, of a substance, including without limitation a therapeutic agent from the dosage form or composition containing the substance.

As used herein, the term “controlled release” refers to non-immediate release of an agent or substance, including a therapeutic agent, from a composition, device, or formulation. Examples of specific types of controlled release include without limitation, extended or sustained release and delayed release. Any number of control mechanisms or components can be used to create a controlled release effect, including formulation ingredients, materials, or constituents, formulation properties or states, such as pH, an environment in which the formulation is placed, or a combination thereof. In one example, extended release can include release of a therapeutic agent at a level that is sufficient to provide a therapeutic effect or treatment for a non-immediate specified or intended duration of time.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 ml to about 80 ml” should also be understood to provide support for the range of “50 ml to 80 ml.” Furthermore, it is to be understood that in this specification support for actual numerical values is provided even when the term “about” is used therewith. For example, the recitation of “about” 30 should be construed as not only providing support for values a little above and a little below 30, but also for the exact numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 1.5, 2, 2.2, 3, 3.8, 4, 4.6, and 5, individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

Reference in this specification may be made to devices, structures, systems, or methods that provide “improved” performance. It is to be understood that unless otherwise stated, such “improvement” is a measure of a benefit obtained based on a comparison to devices, structures, systems or methods in the prior art. Furthermore, it is to be understood that the degree of improved performance may vary between disclosed embodiments and that no equality or consistency in the amount, degree, or realization of improved performance is to be assumed as universally applicable.

### EXAMPLE EMBODIMENTS

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

Maxilla-craniofacial, cranial, spinal, and other bone irregularities can result from birth defects, trauma, disease, and other causes. A subject with such defects can have significant health risks, as well as significant structural and functional limitations. To repair these defects, surgeons can implant structures of rigid material such as polyether ether ketone (PEEK) or rigid metallic materials such as titanium alloys into the defected area to replace missing bone. While these rigid materials provide the structural support needed to reduce some health risks and limitations associated with the defect, they must be left in the patient long-term and can result in a large number of revision surgeries and other complications as the patient's physiology changes.

Alternatively, artificial or natural bone fragments can be anchored into the defect with bone cements. However, bone cements are also very rigid and do not allow sufficient flexibility to adapt to a patient's changing physiology, for example in young patients with growing and developing bones. Further, these bone fragments poorly approximate the defected area, leaving gaps or areas that have to be filled in with additional bone cement.

The use of highly rigid materials and/or bone fragments in osseo-reparative surgeries presents significant limitations. Bone grafting to allow oseogenesis of new bone can be a superior method for osseo-reparative surgeries. However, proper placement and retention of bone grafting media is critical and requires significant precision. Further, the bone grafting materials much have adequate nutrient supply or ossification will be hampered.

Due to the current limitations in osseo-reparative surgeries and other similar surgeries requiring cranial implants, craniofacial implants, spinal implants, and the like, the present inventors have recognized a need for improved implantable devices, systems, methods, and materials to more adequately facilitate osseogenesis and osseointegration. Accordingly, the present disclosure includes various embodiments of implantable devices, systems, and associated methods to facilitate both osseogenesis and osseointegration in osseo-reparative surgeries. Disclosed invention embodiments can provide structural support in the defect area while maintaining device flexibility, immobilizing bone grafting material at a desired location, providing adequate vasculature access to bone grafting media to allow and facilitate oseogenesis, and reducing the need for revision surgeries.

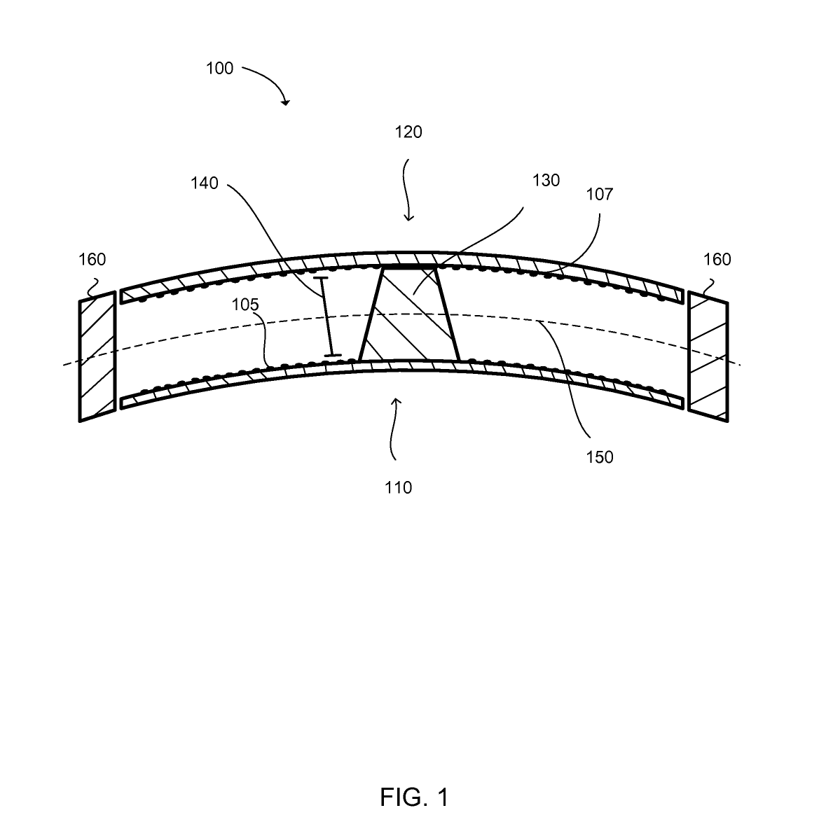

Referring to FIG. 1 there is shown a cross section of an implantable bone grafting device 100 having a base 110, a lid 120, and at least one connector 130 that connects the base 110 and lid 120 in a fixed relationship. There is a space 140 between the base 110 and the lid 120 that is configured as a reservoir to hold, and allow vascular access to, a bone grafting media. The device 100 can also be configured for placement in a subject such that the bone grafting media reservoir substantially aligns in a common plane 150 with a bone 160 of the subject.

The device can be made of any suitable structural material for an implantable bone grafting device 100. Adequate structural materials can be biodegradable materials, non-biodegradable materials, artificial materials, naturally derived materials, and combinations thereof. Additionally, the structural material can include any therapeutic additives suitable to be incorporated or included in or with the implantable device. Some non-exclusive examples of suitable additives can include bone morphogenetic proteins, cytokines, growth factors, matrikines, chemoattractants, anti-inflammatory agents, immunomodulatory agents, antibiotics, glucocorticoids, cytostatics, antibodies, anti-histamines, and combinations thereof.

In one aspect, the structural material can be a non-biodegradable material. Any non-biodegradable materials suitable for use with an implantable device can be used. Some non-exclusive examples of non-biodegradable materials that can be used are ceramics, polymers, metals, alloys, and combinations thereof.

In another aspect, the structural material can be a naturally derived material. Any naturally derived material suitable for use in an implantable device can be used. The naturally derived material can be biodegradable or non-biodegradable. Some non-exclusive examples of naturally derived materials that can be used are extracellular matrix proteins, autologous bone, allografted bone, xenografted bone, decellularized materials, plant-based materials, bacteria-produced materials, and combinations thereof.

In another aspect, the structural material can be a biodegradable material. Any biodegradable material suitable for use in an implantable device can be used. The biodegradable material can be natural or synthetic. Some non-exclusive examples of biodegradable materials that can used are poly(caprolactone), poly(lactic acid), poly(glycolic acid), poly(p-dioxanone), trimethylene carbonate, glycolide, hydroxyapatite, copolymers thereof, synthetic bone, and combinations thereof.

As is well known in the art, suitable biodegradable materials can be adapted to degrade at a predetermined rate or over a predetermined amount of time. Therefore, these materials can be incorporated in the current device to perform these same functions and can be tailored for specific patients, defects, injuries, surgeries, rates of degradation, or the like in order to achieve a specific result. To this end, the materials of the lid and the base can be the same or different. Any combination can be used. In one embodiment, the base and lid can be made of different structural materials. In one aspect, the lid can be made of a biodegradable material and the base can be made of a naturally derived material. In another aspect the lid can be made of a biodegradable material and the base can be made of a non-biodegradable material or an artificial material. In one aspect, the base can be made of a biodegradable material and the lid can be made of a non-biodegradable material or artificial material. In another aspect, the base can be made of a biodegradable material and the lid can be made of a naturally derived material.

In another aspect, the base and the lid can both be made of biodegradable materials. The base and the lid can be configured or adapted to biodegrade at the same rate or at differing rates. Further, both the base and the lid can each be adapted to have sections, areas, features, or components that biodegrade at different rates than other sections, areas, features, or components.

The materials used in the device can allow for zones or areas of expansion. These expansion zones can have various expansion features or structures and in one aspect, can be incorporated by customizing the composition of the structural material based on the stress and strain characteristics of the material. In another aspect, the material can be selected and/or prepared to have targeted stress and strain characteristics. In yet another aspect, expansion zones can be provided by incorporating expandable slots, pressure induced breakage sites, combinations thereof, and the like. In some embodiments, pressure induced breakage sites can be created by providing selected regions with a specific thickness which is sufficiently thin to allow breakage in order to expand. Alternatively, scoring or grooving the material allow for pressure induced breakage. In yet other aspects, the material itself can be varied within the expansion zone to include a material that is easier to break than materials outside the expansion zone.

The base can be of any suitable thickness for an implantable device and can be tailored for a specific subject, surgery, defect, injury, rate of degradation, physiologic placement, bone type, or the like. In one aspect, the base can be from 0.1 mm to 10 mm thick. In another aspect, the base can be from 0.1 mm to 5 mm thick. In another aspect, the base can be from 0.5 mm to 5 mm thick. In one aspect, the base can be from 1 mm to 3 mm thick. In one aspect, the base can have a uniform thickness throughout. In another aspect, the base can have a variety of thicknesses throughout.

Returning to FIG. 1, in one embodiment, the interior of the base 110 can have a textured surface. The textured surface can include a variety of asperities 105. The asperities 105 can be of uniform height or of variable height. In one aspect the asperities 105 can have a height of from about 1 μm to about 10 mm. The texture can have predetermined pattern or a random pattern. Alternatively, the interior surface of the base 110 can be smooth.

Likewise, the lid can be of any suitable thickness for an implantable device and can be tailored for a specific surgery, defect, rate of degradation, physiologic location, procedure, bone type, purpose, or the like. In one aspect, the lid can be from 0.1 mm to 10 mm thick. In another aspect, the lid can be from 0.1 mm to 5 mm thick. In another aspect, the lid can be from 0.5 mm to 5 mm thick. In one aspect, the lid can be from 1 mm to 3 mm thick. In one aspect, the lid can be from 0.5 mm to 1.5 mm thick. In one aspect, the lid can have a uniform thickness throughout. In another aspect, the lid can have a variety of thicknesses throughout.

Returning again to FIG. 1, the interior of the lid 120 can have a textured surface. The textured surface can include a variety of asperities 107 as are found on the lid 120. The asperities 107 can be of uniform height or of variable height. In one aspect the asperities 107 can have a height of from about 1 μm to about 10 mm. The texture can have predetermined pattern or a random pattern. Alternatively, the interior surface of the lid 120 can be smooth. In some aspects, a textured surface may promote or accelerate oseogenesis and reduce scarring.

The base and the lid are configured to be connected in a fixed relationship with one another. However, the perimeters of the base and the lid need not be equivalent. In one aspect, the base has a perimeter that is larger than a perimeter of the lid. In another aspect, the lid has a perimeter that is larger than a perimeter of the base. In another aspect, the lid and the base have substantially matching perimeters.

Whether or not the perimeters of the base and lid have matching shapes or sizes it should be noted that the perimeters can have any geometric shape, whether regular or irregular. In some embodiment, the shape may be a square, circle, rectangle, triangle, hexagon, trapezoid, oval, or any other know geometric shape. In additional embodiments the perimeter can have a custom shape that corresponds or substantially corresponds to a shape of an area (e.g. an area of missing bone) into which the device is to be positioned or placed. Such shapes can be selected using the shape of the space to be filled and can be implemented during fabrication, for example by 3D printing as further discussed below.

Either the base or the lid can be configured to couple to a portion of an adjacent bone via a coupling member or fastener (not shown). Any suitable device or mechanism can be used. Some non-exclusive examples of suitable coupling members or fasteners can include natural adhesives, synthetic adhesives, screws, sutures, staples, pins, tacks, nails, or combinations thereof.

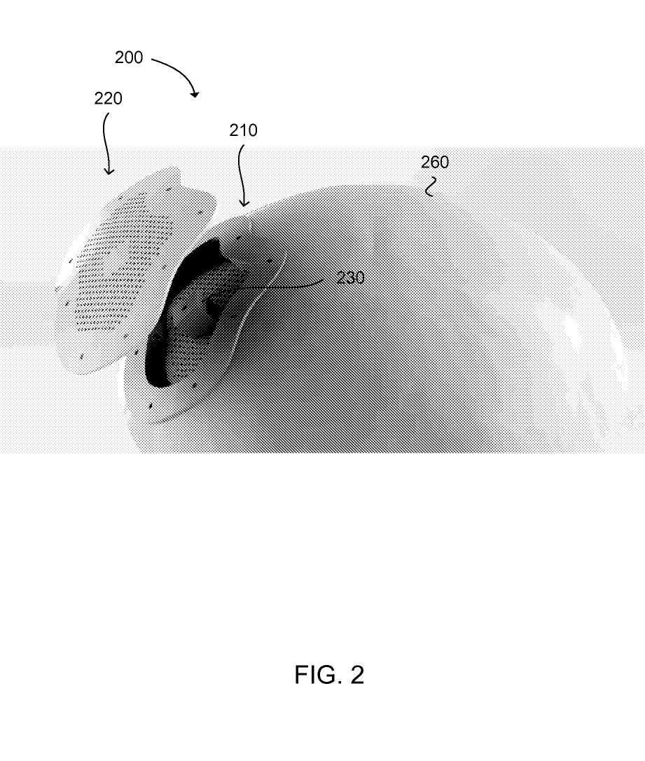

FIG. 2 depicts another embodiment of the current technology. This implantable bone grafting device 200 has a base 210 and a lid 220 with a connector 230 configured to connect the base 210 and lid 220 in a fixed relationship with a space therebetween. The base 210 forms a basket-like structure adapted to hold bone grafting media in a common plane with a bone of a subject. In FIG. 2, the bone is depicted as a skull 260, but this is only one example of a bone that is treatable or with which the current technology can be used.

Both the base 210 and the lid 220 have a plurality of pores formed therein to allow vascular access to the bone grafting media. Though FIG. 2 illustrates an embodiment where both the lid and the base include pores, both need not have pores. In one aspect, only the lid 220 has pores. In another aspect, only the base 210 has pores. In another aspect, as is shown in the FIG. 2 both the lid 220 and the base 210 can have pores. Further, the pores in the lid 220 can be the same shape and/or size as the pores in the base 210, or they can be a different shape and/or size. In another aspect, the lid 220 and the base 210 can each have various sizes of pores. In this case, the lid 220 can also have the same variety of pore sizes as the base 210 or it can have a different variety of pore sizes. In one aspect the vascular access pores can have a pore size of from about 0.2 mm to about 5 mm.

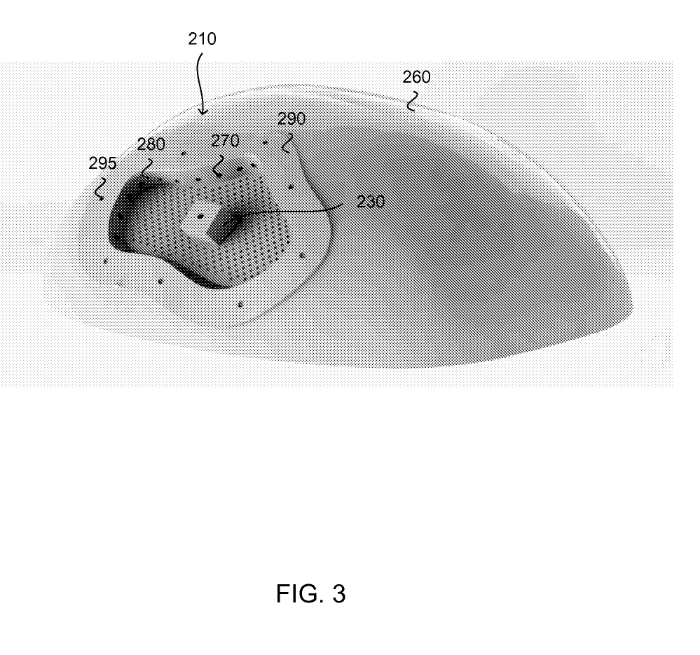

FIG. 3 provides another view of the base 210 aligned in a common plane with a bone 260 of a subject. The base 210 can have a reservoir wall 280 disposed at a perimeter thereof, thus forming a basket-shaped reservoir. The reservoir wall 280 can have pores 270 that are the same sizes as the pores in the rest of the base 210 or that are a different pore size than those found in the rest of the base 210. Similarly the pores 270 in the reservoir wall 280 can have the same pore size or a different pore size than the pores found in the lid (not shown). In this particular embodiment, the base 210 also includes a placement flange 290 disposed on the reservoir wall 280. The placement flange 290 can facilitate the placement of the implantable device at the defect or implant site. In one aspect, the flange overlaps a surface of the bone and rests thereagainst when engaged with the bone. The placement flange 290 can also include a plurality or holes, perforations, or eyelets 295 to assist in securing the device at the defect or implant site.

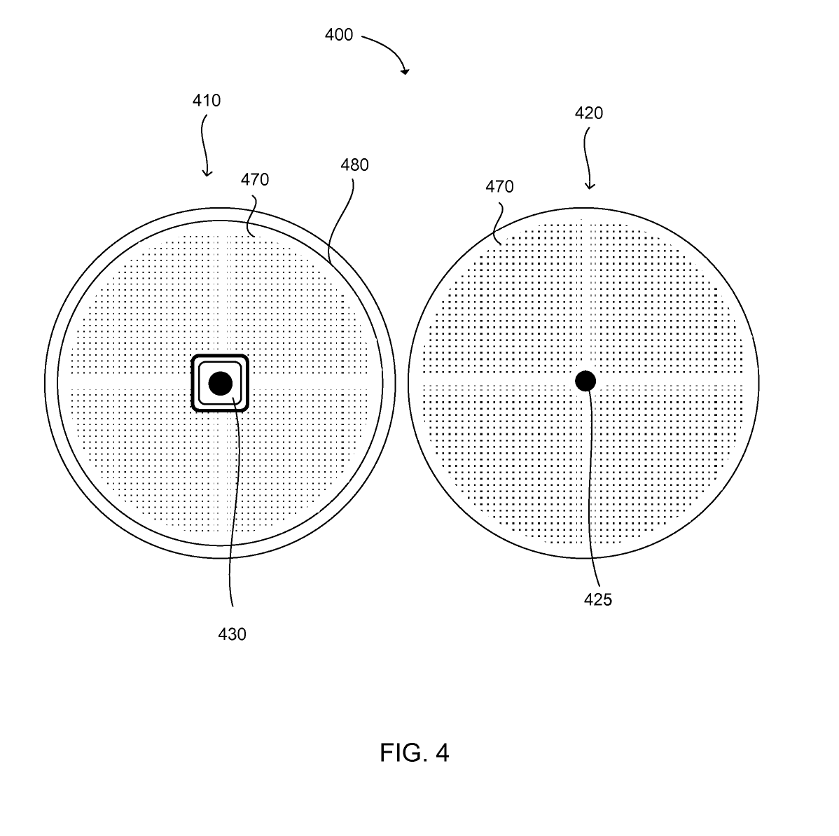

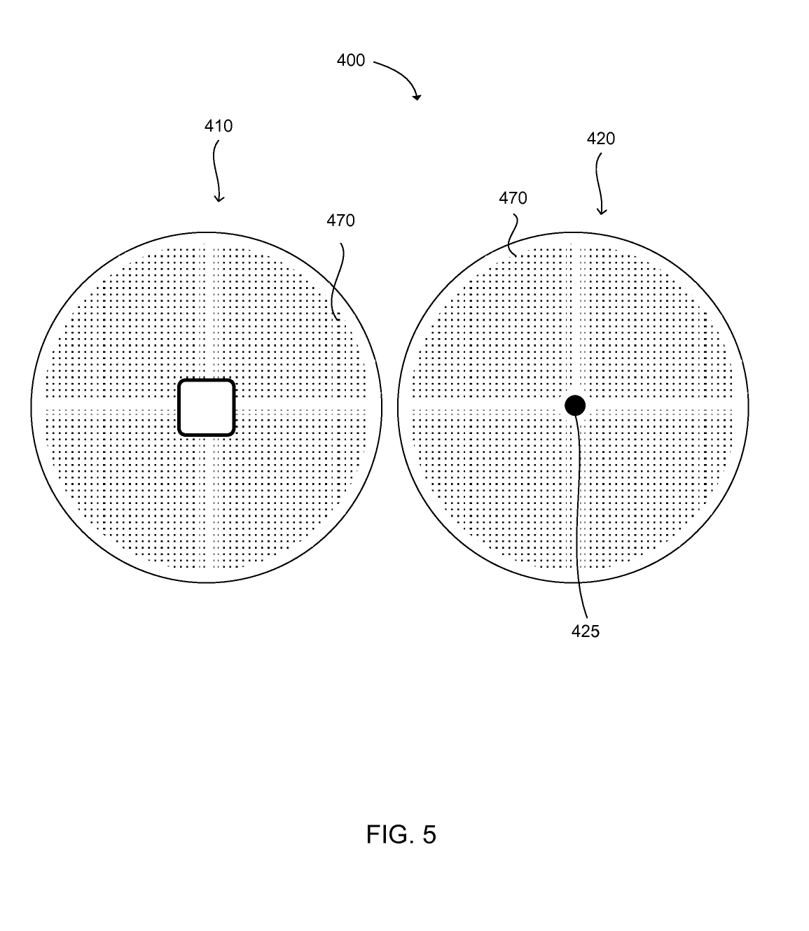

Another embodiment of the implantable bone grafting device 400 is illustrated in FIGS. 4-5. FIG. 4 depicts a view showing the inner surface of the base 410 and the lid 420. The base 410 has a single connector 430 and a reservoir wall 480. The lid 420 has an attachment hole or eyelet 425 to facilitate the coupling of the lid 420 with the base 410 via the connector 430. Additionally, a plurality of mesh-like pores 470 are formed in both the base 410 and the lid 420. The base 410 and the lid 420 each have thicker structural sections to provide greater structural integrity at desired locations. In this particular embodiment, thicker sections are located at a perimeter of both the base 410 and the lid 420 and in a central cruciform section. Additional reinforcements can also be provided as needed. Any suitable reinforcements can be used in connection with the current technology. Some non-limiting examples of additional reinforcement features can include high strength fibers, composite materials, aligned fibers, cross-linking, or any combination thereof.

A view of the outer surface of the base 410 and the lid 420 of bone grafting device 400 is illustrated in FIG. 5. As is illustrated in FIG. 5, the lid 420 has substantially the same appearance on the inner and the outer surface. The attachment hole or eyelet 425 is seen again on the outer surface, which illustrates the pass-through nature of this feature. The outer surface of the base 410 follows the same general shape as the inner surface, but the reservoir wall and the connector cannot be seen. The vascular access pores 470 formed within the base 410 and the lid 420 can be seen on both the inner and outer surfaces of the device 400, which illustrates their pass-through nature.

The device 400 has a very generic, circular shape. However, as can be seen in the various embodiments illustrated herein, the shape of the device can be highly tailored to any suitable shape for an implantable bone grafting device. The device can be tailored based on the patient, the injury, the defect, the surgery, the amount of bone grafting media required or available, desired rates of degradation, and the like.

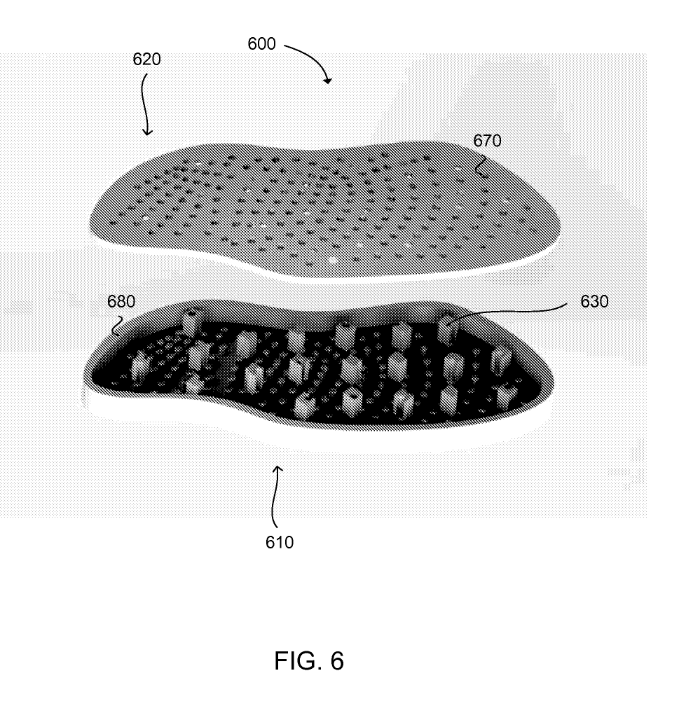

The embodiment in FIGS. 4-5 only includes one connector 430. However, the current technology is not limited to a single connector. Any number of suitable connectors can be used. For example, in the embodiment illustrated in FIG. 6, the implantable bone grafting device 600 has a plurality of connectors 630. The plurality of connectors 630 can connect the base 610 and the lid 620 in a fixed relationship with a space therebetween. In this particular embodiment, the lid 620 has a plurality of pores 670 and the base 610 has a reservoir wall 680 without a placement flange.

The connectors 630 are illustrated as being associated with the base, but they can also be associated with the lid. In one aspect the lid has connectors. In another aspect the base has connectors. In another aspect, both the base and the lid have connectors. The connectors can be integral with the base and/or lid, or the connectors can be separately attached to the base and/or lid. In one aspect, at least one connector can be integral with at least one of the base and/or the lid. In one aspect, at least one connector can be integral with the base. In one aspect, at least one connector can be integral with the lid. In one aspect, both the base and the lid each have at least one connector integral therewith. In another aspect, at least one connector can be attached to the base and/or the lid. The connector can be attached in any suitable manner. Some none-exclusive mechanisms that can be used for attaching the connector can include at least one of alignment screws, fixation screws, interlocking parts, securement tabs, adhesives, photo-curing, thermal-curing, thermosetting, chemical crosslinking, or combinations thereof.

In another aspect, at least one connector is configured to align the base and the lid in a fixed relationship. In another aspect, at least one connector is configured to maintain a space between the base and the lid. The space can be any suitable distance. In one aspect, the space can be a distance between an interior surface of the base and an interior surface of the lid of 20 mm or less. In another aspect, the space can be from about 1 mm to about 20 mm or 2 cm. In another aspect, the space can be 16 mm or less. In one aspect, the space can be 12 mm or less. In one aspect the space can be 8 mm or less. The space can be uniform across the base and the lid or it can vary from one distance to another. In one aspect, the space can taper from the center to the edges, where the distance can taper to either a lesser or greater distance. In another aspect, the distance can vary randomly or in a predetermined manner. The space can be modified based on a specific subject, injury, defect, surgery, amount of bone grafting material, and the like.

The connectors can be configured in a variety of ways. In one aspect, the connectors can have vascular access pores formed therein. The connectors can also be configured to couple the lid and the base together in any suitable manner. In one aspect, the connectors can be configured to interlock with at least one of the base, the lid, another connector, or combinations thereof The connectors can be used in connection with at least one of alignment screws, fixation screws, interlocking parts, securement tabs, adhesives, photo-curing, thermal-curing, thermosetting, chemical crosslinking, or combinations thereof to attach to the base and or the lid and to couple the base and the lid together. The lid and the base can also be coupled together using natural adhesives, synthetic adhesives, fixation screws, sutures, staples, pins, or combinations thereof to engage with the connectors or to be used in addition to the connectors.

Another embodiment of the current technology encompasses methods of administering bone grafting media to a subject. In another embodiment, methods of promoting, inducing, or accelerating oseogenesis are included. Such methods can include locating and containing (or otherwise immobilizing) a bone graft media at an implantation site (e.g. a wound site). In some embodiments, the bone graft media can be non-adhesively held in place. Such methods can additionally include aligning bone graft media in the same, or substantially the same, or a common plane with a bone to which the bone graft is meant to knit. In some embodiments, the bone graft or bone grafting media can be contained at the site by providing and/or using an implantable bone grafting device as recited herein. Bone grafting media can be retained within a reservoir of the implantable device and the implantable device can be implanted at a pre-selected location (e.g. implantation or wound site) within a subject. Additional agents, compounds or additives can be held within the reservoir along with the bone graft media, such as agents and compounds that aid or accelerate oseogenesis, nutrients, etc. The implantation can place the device in a location that allows the reservoir to substantially align and be held in place in a common plane, or a substantially common plane, with a bone selected to receive the bone graft.

Any suitable bone grafting media can be used in connection with this method. Some non-exclusive examples of suitable bone grafting media can include autologous bone, allografted bone, synthetic bone, xenograft bone, and combinations thereof. Additionally, various therapeutic agents can be included with the bone grafting media. Any suitable therapeutic agent can be included. Some non-exclusive examples of therapeutic agents that can be included are bone morphogenetic proteins, cytokines, growth factors, matrikines, chemo-attractants, anti-inflammatory agents, immunomodulatory agents, antibiotics, glucocorticoids, cytostatics, antibodies, anti-histamines, and combinations thereof.

Any suitable subject can benefit from this method. In one specific aspect, the subject can be a human subject. In another aspect, the human can be a child. In a further aspect, the human can be an adolescent. In yet another aspect, the human can be an adult. In one embodiment, the implantation site can be a wound or injury. In another embodiment, the implantation site can be a birth defect. In a further embodiment, the implantation site can be a site that is surgically created or altered.

Providing the implantable bone grafting device as described herein can include customizing the device in any way suitable or necessary to perform the method. The device can be customized based on a specific subject, a specific injury or medical condition, a specific medical procedure, a desired rate of degradation, or combinations thereof. Customization can include any parameter of the implantable bone grafting device. For example, customization can include the device geometry, size, thickness, structural composition, additive composition, rate of biodegradation, or combinations thereof. Additionally, customization can go beyond customizing the device itself. For example, customization can include customization of the bone grafting media used and the manner in which the device is implanted and secured to the subject. As a further example, the additional step of securing the device at the pre-selected location can include any suitable securing mechanism. Such securing mechanisms can include natural adhesives, synthetic adhesives, fixation screws, sutures, staples, pins, or combinations thereof.

The high customization of the device can be based on a CT scan or other suitable imaging of the defect site. Once the defect site is mapped, the device can be customized from appropriate structural materials using any suitable method. Some non-exclusive examples of suitable constructions methods and/or tools can include selective laser sintering, 3D printing, computer numerical control tooling, electro-spinning, extrusion, fused deposition modeling, or other build up or cut down fabrication processes.

Another embodiment of the current technology includes a system for administering bone grafting media to a subject. The system can include an implantable bone grafting device as recited herein and bone grafting media suitable for use with the device. The bone grafting media can include autologous bone, allografted bone, synthetic bone, xenograft bone, and combinations thereof. Therapeutic additives can also be included with the bone grafting media. Any suitable therapeutic additives can be included. Such additives can include bone morphogenetic proteins, cytokines, growth factors, matrikines, chemoattractants, anti-inflammatory agents, immunomodulatory agents, antibiotics, glucocorticoids, cytostatics, antibodies, anti-histamines, and combinations thereof.

### EXAMPLES

Some non-exclusive example embodiments of the current technology include the following:

In one example, there is provided an implantable bone grafting device, comprising a base, a lid, and at least one connector that connects the base and lid in a fixed relationship with a space therebetween, said space configured as a reservoir to hold, and allow vascular access to, a bone grafting media, wherein the implantable device is configured for placement in a subject such that the bone grafting media reservoir substantially aligns in a common plane with a bone of the subject.

In one example, the device comprises a structural material that is a member selected from the group consisting of: biodegradable materials, non-biodegradable materials, artificial materials, naturally derived materials, and combinations thereof.

In one example, the structural material includes a therapeutic additive.

In one example, the therapeutic additive is a member selected from the group consisting of: bone morphogenetic proteins, cytokines, growth factors, matrikines, chemoattractants, anti-inflammatory agents, immunomodulatory agents, antibiotics, glucocorticoids, cytostatics, antibodies, anti-histamines, and combinations thereof.

In one example, the structural material comprises a non-biodegradable material selected from the group consisting of: ceramics, polymers, metals, alloys, and combinations thereof.

In one example, the structural material comprises a naturally derived material selected from the group consisting of: extracellular matrix proteins, autologous bone, allografted bone, xenografted bone, decellularized materials, plant-based materials, bacteria-produced materials, and combinations thereof.

In one example, the structural material comprises a biodegradable material.

In one example, the biodegradable material is selected from the group consisting of poly(caprolactone), poly(lactic acid), poly(glycolic acid), poly(p-dioxanone), trimethylene carbonate, glycolide, hydroxyapatite, copolymers thereof, synthetic bone, lactides, urethanes, ester amides, ortho esters, anhydrides, propylene furmarates, amino acids, cyanoacrylates, phosphazenes, phosphoesters and combinations thereof.

In one example, the biodegradable material is configured to degrade at a predetermined rate or over a predetermined amount of time.

In one example, the structural material in the base differs from the composition of the structural material in the lid.

In one example, the structural materials are configured to biodegrade at different rates.

In one example, the vascular access is provided by pores formed in the device.

In one example, the vascular access pores have a pore size of from 0.2 mm to 5 mm.

In one example, vascular access pores are formed within the base.

In one example, vascular access pores are formed within the lid.

In one example, vascular access pores are formed within the base and the lid.

In one example, the vascular access pores in the base and the lid have different sizes.

In one example, the base further comprises a reservoir wall disposed along a perimeter thereof.

In one example, vascular access pores are formed within the reservoir wall.

In one example, the vascular access pores formed within the reservoir wall have a different pore size than vascular access pores formed within the base and/or the lid.

In one example, a placement flange is disposed on the reservoir wall.

In one example, the base has a thickness of from about 0.1 mm to about 10 mm.

In one example, the lid has a thickness of from about 0.1 mm to about 10 mm.

In one example, the at least one connector is integral with at least one of the base or the lid.

In one example the at least one connector is integral with the base.

In one example, the at least one connector is integral with the lid.

In one example, both the base and the lid each have at least one connector integral therewith.

In one example, the at least one connector is configured to align the base and the lid in the fixed relationship.

In one example, the at least one connector is configured to maintain the space between the base and the lid.

In one example, vascular access pores are formed within the at least one connector.

In one example, the at least one connector is configured to interlock with at least one of the base, the lid, another connector, or combinations thereof.

In one example, the at least one connector attaches to the base and the lid via at least one of alignment screws, fixation screws, interlocking parts, securement tabs, adhesives, photo-curing, thermal-curing, thermosetting, chemical crosslinking, or combinations thereof.

In one example, the space between the base and the lid is a distance of 20 mm or less.

In one example, the lid and the base are coupled using a coupling member selected from the group consisting of: natural adhesives, synthetic adhesives, fixation screws, sutures, staples, pins, or combinations thereof.

In one example, either the lid or the base is configured to couple to a portion of an adjacent bone with a coupling member.

In one example, the coupling member is selected from the group consisting of: natural adhesives, synthetic adhesives, fixation screws, sutures, staples, pins, or combinations thereof.

In one example, the reservoir has a width between an interior surface of the lid and an interior surface of the base of from about 1 mm to about 2 cm.

In one example, the base has a perimeter that is larger than a perimeter of the lid.

In one example, the lid has a perimeter that is larger than a perimeter of the base.

In one example, the lid and the base have substantially matching perimeters.

In one example, at least a portion of an interior surface of the reservoir is smooth.

In one example, at least a portion of an interior surface of the reservoir is textured.

In one example, the texture includes asperities having a height of from about 1 um to about 10 mm.

In one example, the texture has a predetermined pattern.

In one example, the texture is random.

In one example there is provided a method of administering bone grafting media to a subject, comprising providing an implantable bone grafting device as recited herein, retaining bone grafting media within a reservoir of the implantable device, and implanting the implantable device at a pre-selected location within the subject so that the reservoir substantially aligns and is held in place in a common plane with the bone selected to receive the bone graft.

In one example, the bone grafting media is selected from the group consisting of autologous bone, allografted bone, synthetic bone, xenograft bone, and combinations thereof.

In one example, the method further comprises including a therapeutic additive in the bone graft media.

In one example, the therapeutic additive is selected from the group consisting of bone morphogenetic proteins, cytokines, growth factors, matrikines, chemoattractants, anti-inflammatory agents, immunomodulatory agents, antibiotics, glucocorticoids, cytostatics, antibodies, anti-histamines, and combinations thereof.

In one example, the subject is a human.

In one example, the step of providing includes customizing the device for a specific subject, a specific injury or medical condition, a specific medical procedure, or combinations thereof.

In one example, customizing includes customizing the device geometry, size, thickness, composition, rate of biodegradation, or combinations thereof.

In one example, the device is secured at the pre-selected location via natural adhesives, synthetic adhesives, fixation screws, sutures, staples, pins, or combinations thereof.

In one example, there is provided a system for administering bone grafting media to a subject, comprising an implantable bone grafting device as previously described and a bone grafting media suitable for use with the implantable bone grafting device.

In one example, the bone grafting media is selected from the group consisting of autologous bone, allografted bone, synthetic bone, xenograft bone, and combinations thereof.

In one example, the bone grafting media includes a therapeutic additive.

In one example, the therapeutic additive is selected from the group consisting of bone morphogenetic proteins, cytokines, growth factors, matrikines, chemoattractants, anti-inflammatory agents, immunomodulatory agents, antibiotics, glucocorticoids, cytostatics, antibodies, anti-histamines, and combinations thereof.

While the forgoing examples are illustrative of the specific embodiments in one or more particular applications, it will be apparent to those of ordinary skill in the art that numerous modifications in form, usage and details of implementation can be made without departing from the principles and concepts articulated herein. Accordingly, no limitation is intended except as by the claims set forth below.

1. An implantable bone grafting device, comprising:
a base;
a lid; and
at least one connector that connects the base and lid in a fixed relationship with a space therebetween, said space configured as a reservoir to hold, and allow vascular access to, a bone grafting media;
wherein the implantable device is configured for placement in a subject such that the bone grafting media reservoir substantially aligns in a common plane with a bone of the subject.

2. The device of claim 1, wherein the device comprises a structural material that is a member selected from the group consisting of: biodegradable materials, non-biodegradable materials, artificial materials, naturally derived materials, and combinations thereof.

3-12. (canceled)

13. The device of claim 1, wherein the vascular access is provided by pores formed in the device.

14-18. (canceled)

19. The device of claim 1, wherein the base further comprises a reservoir wall disposed along a perimeter thereof.

20-22. (canceled)

23. The device of claim 1, wherein the base has a thickness of from about 0.1 mm to about 10 mm.

24. The device of claim 1, wherein the lid has a thickness of from about 0.1 mm to about 10 mm.

25. The device of claim 1, wherein the at least one connector is integral with at least one of the base or the lid.

26-33. (canceled)

34. The device of claim 1, wherein the space between the base and the lid is a distance of 20 mm or less.

35. The device of claim 1, wherein the lid and the base are coupled using a coupling member selected from the group consisting of: natural adhesives, synthetic adhesives, fixation screws, sutures, staples, pins, or combinations thereof.

36. The device of claim 1, wherein either the lid or the base is configured to couple to a portion of an adjacent bone with a coupling member.

37. (canceled)

38. The device of claim 1, wherein the reservoir has a width between an interior surface of the lid and an interior surface of the base of from about 1 mm to about 2 cm.

39. The device of claim 1, wherein the base has a perimeter that is larger than a perimeter of the lid.

40. The device of claim 1, wherein the lid has a perimeter that is larger than a perimeter of the base.

41. The device of claim 1, wherein the lid and the base have substantially matching perimeters.

42. The device of claim 1, wherein at least a portion of an interior surface of the reservoir is smooth.

43. The device of claim 1, wherein at least a portion of an interior surface of the reservoir is textured.

44-46. (canceled)

47. The device of claim 1, wherein the lid and/or the base include an expansion zone.

48. (canceled)

49. A method of administering bone grafting media to a subject, comprising:
providing an implantable bone grafting device as recited in claim 1;
retaining bone grafting media within a reservoir of the implantable device; and
implanting the implantable device at a pre-selected location within the subject so that the reservoir substantially aligns and is held in place in a common plane with the bone selected to receive the bone graft.

50. The method of claim 48, wherein the bone grafting media is selected from the group consisting of autologous bone, allografted bone, synthetic bone, xenograft bone, and combinations thereof.

51. The method of claim 48, further comprising including a therapeutic additive in the bone graft media.

52. (canceled)

53. The method of claim 48, wherein the subject is a human.

54. The method of claim 48, wherein the step of providing includes customizing the device for a specific subject, a specific injury or medical condition, a specific medical procedure, or combinations thereof.

55. (canceled)

56. The method of claim 48, wherein the device is secured at the pre-selected location via natural adhesives, synthetic adhesives, fixation screws, sutures, staples, pins, or combinations thereof.

57. A system for administering bone grafting media to a subject, comprising:
an implantable bone grafting device as recited in claims 1; and
a bone grafting media suitable for use with the implantable bone grafting device.

58. The system of claim 56, wherein the bone grafting media is selected from the group consisting of autologous bone, allografted bone, synthetic bone, xenograft bone, and combinations thereof.

59-60. (canceled)
